(19) United States
(12) Patent Application Publication
(10) Pub. No.: US 2016/0029955 A1
(43) Pub. Date: Feb. 4, 2016

(54) ELECTRONIC DEVICE

(71) Applicant: KABUSHIKI KAISHA TOSHIBA, Tokyo (JP)

(72) Inventor: Koichi Sato, Tokyo (JP)

(73) Assignee: KABUSHIKI KAISHA TOSHIBA, Tokyo (JP)

(21) Appl. No.: 14/883,080

(22) Filed: Oct. 14, 2015

Related U.S. Application Data
(63) Continuation of application No. PCT/JP2013/069178, filed on Jul. 12, 2013.

(51) Int. Cl. A61B 5/00; H04B 1/3827; A61B 5/04; H04W 4/00

(52) U.S. Cl. CPC A61B 5/681; H04W 4/008; H04B 1/385; A61B 5/04017; A61B 5/742

(57) ABSTRACT

According to one embodiment, an electronic device includes a main body and a band. The main body includes a vital-signs sensor and a short-range communicator. The band is configured to attach the main body to a human body. At least one first electrode of two or more electrodes for the vital-signs sensor is provided on a surface of the band on a side in contact with the human body, and is used as a coupling portion for the short-range communicator.

### CROSS REFERENCE TO RELATED APPLICATIONS

This application is a Continuation Application of PCT Application No. PCT/JP2013/069178, filed Jul. 12, 2013, the entire contents of which are incorporated herein by reference.

### FIELD

Embodiments described herein relate generally to an electronic device.

### BACKGROUND

In recent years, battery-powered portable electronic devices such as tablets and smartphones are widely used. Recently, electronic devices called, for example, wearable devices, worn on the human body like a wristwatch or eyeglasses have also appeared.

This kind of electronic devices often have a short-range communication function of communicating with another device only by performing acts such as holding the electronic device over the other device or touching the other device with the electronic device. Various proposals have been previously advanced on the short-range communication function.

In order to exploit the characteristics of being worn on the human body, it is effective to equip the wearable device with a vital-signs sensor which acquires physiological information such as an electrocardiogram. Meanwhile, size and weight reduction are strongly required because of the property of being worn on the human body.

For this reason, it has been difficult to secure space to accommodate a coupling portion (coil, antenna, electrode, etc.) for a short-range communication module (to provide the above-described short-range communication function) and electrodes for the vital-signs sensor.

### BRIEF DESCRIPTION OF THE DRAWINGS

A general architecture that implements the various features of the embodiments will now be described with reference to the drawings. The drawings and the associated descriptions are provided to illustrate the embodiments and not to limit the scope of the invention.

FIG. 1 is an exemplary perspective view of an electronic device of the first embodiment.

FIG. 2 is an exemplary first illustration of an example of mounting a coupling portion for a short-range communication module and electrodes for a vital-signs sensor in the electronic device of the first embodiment.

FIG. 3 is an exemplary second illustration of the example of mounting the coupling portion for the short-range communication module and the electrodes for the vital-signs sensor in the electronic device of the first embodiment.

FIG. 4 is an exemplary block diagram showing a mechanism regarding the mounting of the short-range communication module and the vital-signs sensor of the electronic device of the first embodiment.

FIG. 5 is an exemplary first illustration of an example of mounting an antenna for a wireless communication module in an electronic device of the second embodiment.

FIG. 6 is an exemplary second illustration of the example of mounting the antenna for the wireless communication module in the electronic device of the second embodiment.

### DETAILED DESCRIPTION

Various embodiments will be described hereinafter with reference to the accompanying drawings.

In general, according to one embodiment, an electronic device includes a main body and a band. The main body includes a vital-signs sensor and a short-range communicator. The band is configured to attach the main body to a human body. At least one first electrode of two or more electrodes for the vital-signs sensor is provided on a surface of the band on a side in contact with the human body, and is used as a coupling portion for the short-range communicator.

### First Embodiment

First, the first embodiment is described.

An electronic device of the present embodiment is implemented as a so-called wearable device worn on a human body. In the description below, the electronic device is assumed to be implemented as a wristwatch-type wearable device.

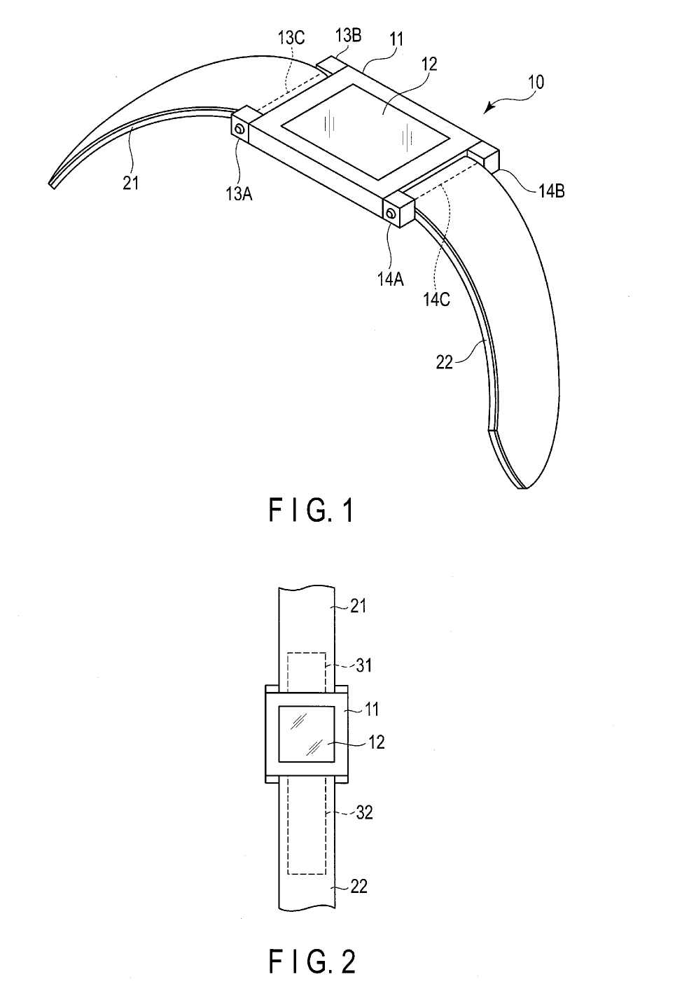

FIG. 1 is an exemplary perspective view of a wearable device 10. The wearable device 10 comprises a main body 11. The main body 11 is constituted by a thin housing. Various electronic components are provided in the housing. A display 12 such as a liquid crystal display (LCD) is placed on the upper surface of the main body 11. The display 12 may be a touchscreen display capable of detecting a contact position on the display screen.

The wearable device 10 comprises a band capable of attaching the main body 11 to the human body (arm). The term “band” is synonymous with a belt. FIG. 1 exemplarily shows a case where the band is divided into two bands 21 and 22, but the band is not limited to this. For example, bands 21 and 22 may be different portions of the single band. Each of bands 21 and 22 is realized by a member having flexibility such as leather.

One end of band 21 is attached to, for example, one side surface (for example, an upper side surface) of the main body 11. Two attachment portions 13A and 13B are placed on the upper side surface of the main body 11 so as to protrude from the upper side surface of the main body 11. The one end of band 21 is wound around a rod 13C supported by the two attachment portions 13A and 13B.

One end of band 22 is attached to, for example, another side surface (for example, a lower side surface) of the main body 11 opposed to the one side surface described above. Two attachment portions 14A and 14B are placed on the lower side surface of the main body 11 so as to protrude from the lower side surface on the main body 11. The one end of band 21 is wound around a rod 14C supported by the two attachment portions 14A and 14B.

As the appearance is shown in FIG. 1, the wearable device 10 of the present embodiment is an electronic device used by attaching the main body 11 to the human body (arm) by bands 21 and 22. Therefore, size and weight reduction of the main body 11 are required. In other words, space to accommodate the electronic components is limited. Meanwhile, the wearable device 10 is equipped with a vital-signs sensor which acquires physiological information such as an electrocardiogram to exploit the characteristics of being worn on the human body (arm), in addition to a short-range communication module which performs, for example, communication conforming to the Near-Field Communication (NFC) standard, or human body communication. Communication conforming to the NFC standard is communication using electromagnetic induction. Human body communication is communication using electric field coupling (weak electric field generated on the surface of the human body), i.e., communication using the human body as a communication channel. The wearable device 10 implements the placement of a coupling portion (NFC: coil/antenna; human body communication: electrode) for the short-range communication module and electrodes for the vital-signs sensor under the condition that space to place the electronic components is limited. The detailed description is hereinafter provided in this regard.

FIG. 2 is an exemplary illustration of an example of mounting the coupling portion for the short-range communication module and the electrodes for the vital-signs sensor in the wearable device 10.

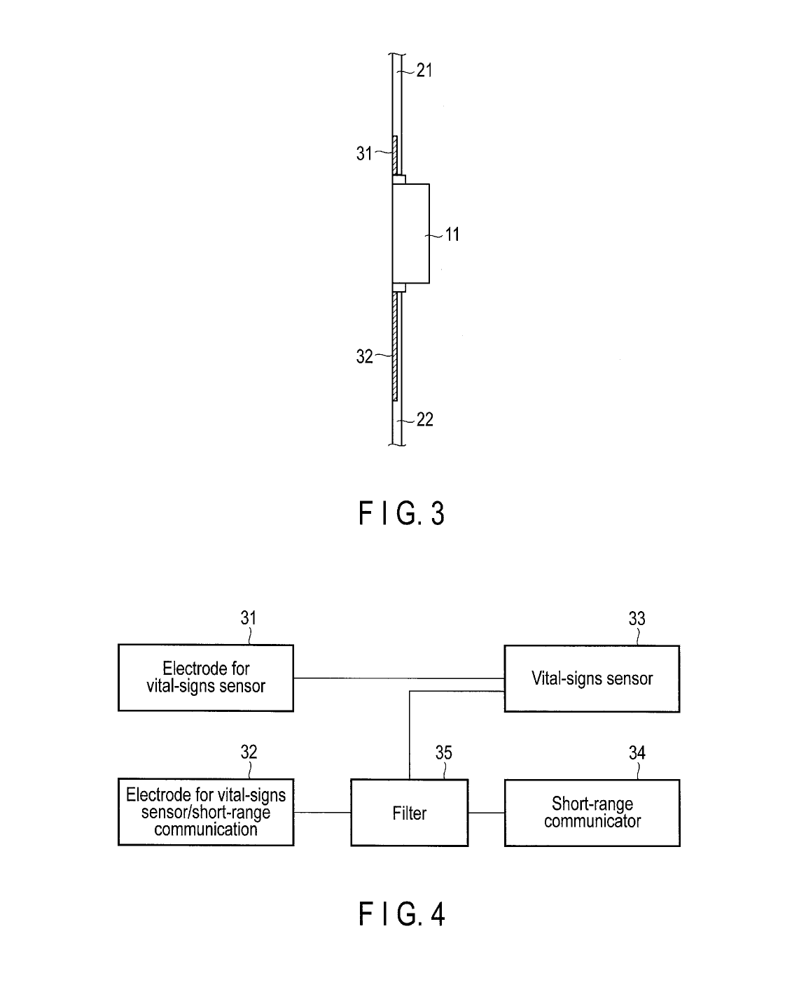

As shown in FIG. 2, in the wearable device 10, electrodes 31 and 32 for the vital-signs sensor are mounted on bands 21 and 22. As shown in FIG. 3, electrodes 31 and 32 are placed on the inner surfaces (in contact with the human body) of bands 21 and 22. In the wearable device 10, electrode 32 for the vital-signs sensor placed on band 22 is also used as a coupling portion for the short-range communication module.

Furthermore, in the wearable device 10, a filter for separating signals is provided between electrode 32, and the short-range communication module and the vital-signs sensor. The filter is mounted in the main body 11. FIG. 4 is an exemplary block diagram showing a mechanism regarding the mounting of the short-range communication module and the vital-signs sensor of the wearable device 10.

As shown in FIG. 4, the wearable device 10 comprises electrode 31 for the vital-signs sensor, electrode 32 for the vital-signs sensor and the short-range communication, the vital-signs sensor 33, the short-range communicator 34 and the filter 35.

The vital-signs sensor 33 acquires the physiological information of the user based on a differential signal obtained by the two electrodes 31 and 32. The short-range communicator 34 performs short-range communication using electrode 32 of electrodes 31 and 32 for the vital-signs sensor 33. In order to share electrode 32 between the vital-signs sensor 33 and the short-range communicator 34, the filter 35 for separating signals is provided between electrode 32, and the vital-signs sensor 33 and the short-range communicator 34. For example, the filter 35 is a module that passes a signal in a frequency band of 13.56 MHz to the side of the short-range communicator 34.

As described above, the wearable device 10 can place the electrodes for the vital-signs sensor 33 and the coupling portion for the short-range communicator 34 while conserving space by the shared use of the electrode for the vital-signs sensor 33 and the coupling portion for the short-range communicator 34. In particular, when the short-range communicator 34 performs the human body communication using electric field coupling, it is expected that stable communication can be performed by using the electrode of the vital-signs sensor 33 placed to contact the human body (arm) as the coupling portion. It is also expected to prevent performance degradation of the coupling portion of the short-range communicator 34 by mounting the electrode for the vital-signs sensor 33, which is also used as the coupling portion for the short-range communicator 34, on the band which can secure the distance from the main body 11 having a high proportion of metallic components. In the above example, both the two electrodes for the vital-signs sensor 33 are mounted on the band. However, an electrode not used as the coupling portion for the short-range communicator 34 may be placed on, for example, the bottom surface (in contact with the human body) of the main body 11.

### Second Embodiment

Next, the second embodiment is described.

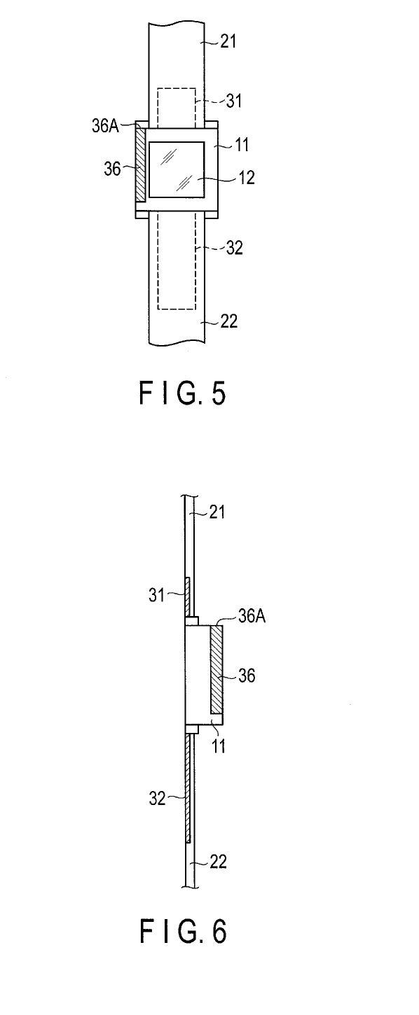

A wearable device 10 of the present embodiment is further equipped with a wireless communication module which performs wireless communication conforming to, for example, the Bluetooth (registered trademark) standard. In the wearable device 10 of the present embodiment, an antenna 36 for the wireless communication module is placed on the upper surface of the main body 11 (on the periphery of the display 12), as shown in FIG. 5. In this case, the antenna 36 is placed such that an open end 36A of the antenna 36 is located on an end of the upper surface of the main body 11 opposite to the side on which electrode 32 also used as the coupling portion for the short-range communicator 34 is provided. The entire antenna 36 should preferably be located in one of the sides of the main body 11 orthogonal to the side in which electrode 32 is placed as shown in FIG. 5, but may be located in the opposite side of the side in which electrode 32 is placed (i.e., the side in which electrode 31 is placed).

It is thereby expected to prevent performance degradation of the antenna 36 for the wireless communication module due to interference with the coupling portion for the short-range communicator 34. Furthermore, it can also be expected to suppress wireless performance degradation by the human body by placing the antenna 36 on the upper surface of the main body 11 since the distance (in particular, of the open end 36A of the antenna 36) from the human body can be secured in comparison with bands 21 and 22, as shown in FIG. 6.

In the present embodiment, the placement of the electrode for the vital-signs sensor 33 also used as the coupling portion for the short-range communicator 34 and the antenna for the wireless communication module is described. However, this relationship of placement is also effective in the placement of the coupling portion for the short-range communicator 34 and the antenna for the wireless communication module in the case where the wearable device 10 is not equipped with the vital-signs sensor. Furthermore, the relationship of placement is effective even when the wearable device 10 is not equipped with the near-field communicator 34.

As described above, according to the wearable device 10 of each embodiment, the placement of the coupling portion for the short-range communication module and the electrodes for the vital-signs sensor can be implemented under the condition that space to place the electronic components is limited.

The various modules of the systems described herein can be implemented as software applications, hardware and/or software modules, or components on one or more computers, such as servers. While the various modules are illustrated separately, they may share some or all of the same underlying logic or code.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An electronic device comprising:
a main body comprising a vital-signs sensor and a short-range communicator; and
a band configured to attach the main body to a human body,
wherein at least one first electrode of two or more electrodes for the vital-signs sensor is provided on a surface of the band on a side in contact with the human body, and is used as a coupling portion for the short-range communicator.

2. The electronic device of claim 1, further comprising a filter configured to separate a signal for the vital-signs sensor and a signal for the short-range communicator, wherein the filter is provided on the main body so as to be located between the first electrode, and the vital-signs sensor and the short-range communicator.

3. The electronic device of claim 1, wherein the coupling portion for the short-range communicator comprises a coil for performing communication using electromagnetic induction.

4. The electronic device of claim 1, wherein the coupling portion for the short-range communicator comprises an electrode for performing communication using electric field coupling.

5. The electronic device of claim 1, wherein:
the main body comprises:
a display device; and
a wireless communicator configured to perform wireless communication using radio waves;
a display of the display device is placed on a surface of the main body on a side facing the human body when the main body is attached to the human body; and
an antenna for the wireless communicator is placed in the main body such that an open end is located at an end of the surface on which the display is provided, the end being opposed to a side on which the first electrode is provided.

6. An electronic device comprising:
a main body comprising a short-range communicator and a wireless communicator; and
a band configured to attach the main body to a human body, wherein:
a coupling portion for the short-range communicator is placed on the band; and
an antenna for the wireless communicator is placed in the main body such that an open end is located at an end of the surface of the main body on a side facing the human body when the main body is attached to the human body.

7. The electronic device of claim 6, wherein the coupling portion for the short-range communicator comprises a coil for performing communication using electromagnetic induction.

8. The electronic device of claim 6, wherein the coupling portion for the short-range communicator comprises an electrode for performing communication using electric field coupling.
